(19) United States
(12) Patent Application Publication
(10) Pub. No.: US 2020/0352913 A1
(43) Pub. Date: Nov. 12, 2020

(54) NEW METHOD FOR TREATING DENGUE VIRUS INFECTION

(71) Applicants: INSERM (INSTITUT NATIONAL DE LA SANTÉ ET DE LA RECHERCHE MÉDICALE), Paris (FR); UNIVERSITÉ DE PARIS, Paris (FR); CENTRE NATIONAL DE LA RECHERCHE SCIENTIFIQUE (CNRS), Paris (FR)

(72) Inventors: Ali AMARA, Paris (FR); Laurent MEERTENS, Paris (FR); Mohamed HAFIRASSOU, Paris (FR)

(21) Appl. No.: 16/765,365

(22) PCT Filed: Nov. 22, 2018
(86) PCT No.: PCT/EP2018/082263

§ 371 Date: May 19, 2020

(30) Foreign Application Priority Data
Nov. 23, 2017 (EP) 17306624.2

(51) Int. Cl. A61K 31/427; A61P 31/14

(52) U.S. Cl. CPC A61K 31/427; A61P 31/14

(57) ABSTRACT

The present invention relates to the treatment of Dengue virus infection. To gain insight into the molecular and cellular function of the DENV RC, the inventors generated a tagged NS1 DENV replicon in order to identify associated host proteins during active viral replication. This allowed an unprecedented mapping of the NS1-host interactome in a relevant system and the identification of cellular modules targeted by the DENV RC. By combining 10 these proteomics data with gene silencing experiments, they identified a set of Host Dependency Factors (HDFs) and Host Restriction Factors (HRFs) that critically impact DENV infection. More they tested the NGI-1 molecule for its OST complex inhibition properties and showed that this molecule can be used to treat Dengue virus infection. Thus, the invention relates to an inhibitor of the OST complex and/or of the CCT complex and/or of 15 RACK1 for use in the treatment of dengue virus infection in a subject in need thereof.

### FIELD OF THE INVENTION

The present invention relates to an inhibitor of the OST complex and/or of the CCT complex and/or of RACK1 for use in the treatment of dengue virus infection in a subject in need thereof.

### BACKGROUND OF THE INVENTION

Dengue virus (DENV) belongs to the flavivirus genus, which encompasses major human pathogens such as yellow fever virus (YFV), West Nile virus (WNV), and ZIKA virus (ZIKV) (Holbrook, 2017). DENV is transmitted to humans by the mosquito vector Aedes aegypti and is the most prevalent arbovirus in tropical and subtropical areas. There are nearly 390 million DENV infections yearly worldwide and up to 96 million dengue cases (Bhatt et al., 2013). Although DENV infections are frequently asymptomatic, they can cause disease ranging from mild fever to fatal dengue hemorrhagic fever and dengue shock syndrome (Guzman and Harris, 2015). Despite decades of research, there is currently no therapy against DENV infection and the recently licensed vaccine provides incomplete protection against the four antigenically distinct DENV serotypes (DENV-1-4) (Capeding et al., 2014).

DENV is an enveloped virus containing a positive-stranded RNA genome of ˜11-kb. Upon entry into the host cell, the viral genome is released and translated by the host cell machinery into a large polyprotein precursor. The latter is processed by host and viral proteases into three structural proteins C (core), prM (precursor of the M protein) and E (envelope) glycoproteins and seven non-structural proteins (NS) NS1, NS2A, NS2B, NS3, NS4A, NS4B and NS5 (Acosta et al., 2014). The structural proteins form the virus particles whereas the NS proteins play a central role in viral replication, assembly and the modulation of innate immune responses (Zeidler et al., 2017). Like all flaviviruses, DENV genome replication takes place within virus-induced vesicles (Ve) derived from the endoplasmic reticulum (ER) membrane invaginations (Miller and Krijnse-Locker, 2008; Welsch et al., 2009). These structures consist of 90 nm-wide vesicles containing a ±11 nm pore that allows exchange between the Ve lumen and the cytosol (Welsch et al., 2009). Within the Ve, the viral NS proteins, viral RNA (vRNA) and some host cell factors form the viral replication complex (RC). DENV also induces ER-derived convoluted membranes (CM), which are physically connected to elongated mitochondria (Chatel-Chaix et al., 2016). Whereas CM are thought to be the site of polyprotein processing (Westaway et al., 1997), the role of these structures during DENV replication remains to be further clarified.

DENV RC formation is tightly coordinated and driven by NS protein self-interactions and viral protein-host factor interactions (Apte-Sengupta et al., 2014). Despite intensive investigations, little is known about the nature of the host cell factors that compose the DENV RC. Several studies have been performed to characterize the NS interacting partners and include yeast two-hybrid (Y2H) screens (Khadka et al., 2011; Le Breton et al., 2011; Limjindaporn et al., 2009; Mairiang et al., 2013; Xu et al., 2011), computational prediction (Doolittle and Gomez, 2011) and immunoprecipitation-coupled mass spectrometry of individually overexpressed viral proteins (Carpp et al., 2014; Dechtawewat et al., 2016). These approaches have been valuable to identify a number of important cellular factors targeted by the viral proteins. However, the NS proteins do not work individually but rather interact with each other and cooperate to assemble into dynamic molecular complexes forming the RC. Thus, it is likely that these previous studies do not fully describe the complete picture of DENV-host interactions occurring in the natural course of infection.

NS1 is an enigmatic glycoprotein that is exclusively encoded by members of the flavivirus genus within the Flaviviridae family and accomplishes different functions during DENV infection. NS1 is a viral toxin that is secreted as hexameric lipoprotein involved in immune evasion and viral pathogenesis (Gutsche et al., 2011; Watterson et al., 2016). NS1 also plays a central role in viral particle production by interacting with the E and prM proteins during assembly in the ER lumen (Scaturro et al., 2015). Perhaps the most striking function played by NS1 is its role as a cofactor during flavivirus replication (Lindenbach and Rice, 1997; Westaway et al., 1997; Youn et al., 2013). Indeed, NS1 forms dimers at the ER lumen, where it localizes with the DENV RC possibly through interactions with NS4A and NS4B (Lindenbach and Rice, 1999; Youn et al., 2012) and is required for an early step of vRNA replication. The atomic structures of DENV and WNV NS1 provided valuable information about protein organization and dimer formation, and identified a distinct domain through which NS1 dimer associates to the ER membrane (Akey et al., 2014). However, the molecular details of NS1 function in viral RNA amplification remain obscure.

### SUMMARY OF THE INVENTION

To gain insight into the molecular and cellular function of the DENV RC, the inventors generated a tagged NS1 DENV replicon in order to identify associated host proteins during active viral replication. This allowed an unprecedented mapping of the NS1-host interactome in a relevant system and the identification of cellular modules targeted by the DENV RC. By combining these proteomics data with gene silencing experiments, they identified a set of Host Dependency Factors (HDFs) and Host Restriction Factors (HRFs) that critically impact DENV infection.

Thus the present invention relates to an inhibitor of the OST complex and/or of the CCT complex and/or of RACK1 for use in the treatment of dengue virus infection in a subject in need thereof.

### DETAILED DESCRIPTION OF THE INVENTION

### Compounds of the Invention and Use Thereof

A first aspect of the invention relates to an inhibitor of the OST complex and/or of the CCT complex and/or of RACK1 for use in the treatment of dengue virus infection in a subject in need thereof.

In one embodiment, the invention relates to an inhibitor of the OST complex for use in the treatment of dengue virus infection in a subject in need thereof.

In one embodiment, the invention relates to an inhibitor of the CCT complex for use in the treatment of dengue virus infection in a subject in need thereof.

In one embodiment, the invention relates to an inhibitor of RACK1 complex for use in the treatment of dengue virus infection in a subject in need thereof.

As used herein the term “OST complex” has its general meaning in the art and denotes a membrane protein complex that transfers a 14-sugar oligosaccharide from dolichol to nascent protein. It is a type of glycosyltransferase. OST is a component of the translocon in the endoplasmic reticulum (ER) membrane. A lipid-linked core-oligosaccharide is assembled at the membrane of the endoplasmic reticulum and transferred to selected asparagine residues of nascent polypeptide chains by the oligosaccharyl transferase complex. The active site of OST is located about 4 nm from the lumenal face of the ER membrane. The proteins which form the OST complex are: STT3A, STT3B, DDOST, RPN1, RPN2, DAD1 and OST4).

As used herein the term “CCT complex” for “cytosolic chaperonin-containing T” has its general meaning in the art and denotes a complex which consists of two identical stacked rings, each containing eight different proteins. Unfolded polypeptides enter the central cavity of the complex and are folded in an ATP-dependent manner. The complex folds various proteins, including actin and tubulin. Alternate transcriptional splice variants of this gene, encoding different isoforms, have been characterized. The proteins which form the CCT complex are: TCP1, CCT2, CCT3, CCT4 and CCT5.

For review about these different complexes see: Ruiz-Canada, C et al, 2009 and Cherepanova et al., 2016.

As used herein the term “RACK1” also known as “guanine nucleotide-binding protein subunit beta-2-like 1 (GNB2L1)” has its general meaning in the art and denotes a 32 kDa protein which interact with lot of proteins like AGTRAP.

As used herein, the terms “inhibitor of the OST complex” or “inhibitor of the CCT complex” or “inhibitor of the RACK1” denotes molecules or compound which can inhibit the activity of the proteins or a molecule or compound which destabilizes the proteins. For example, for the OST complex, an inhibitor of the OST complex will inhibit the transfer of the 14-sugar oligosaccharide from dolichol to nascent protein. The term “inhibitor of the OST complex” or “inhibitor of the CCT complex” or “inhibitor of the RACK1” also denotes inhibitors of the expression of the genes coding for the proteins of the complex OST and CCT or of the gene coding for the protein RACK1. Particularly, the inhibitors of the invention are not cytotoxic, have antiviral properties and have not deleterious effects on cells. To test new potential inhibitors, inhibition of Dengue infection in vitro could be done (see an example of the test in the results part).

According to the invention, an inhibitor of the OST complex denotes also an inhibitor of the proteins of the complex that is to say of the proteins STT3A, STT3B, DDOST, RPN1, RPN2, DAD1 and OST4.

According to the invention, an inhibitor of the CCT complex denotes also an inhibitor of the proteins of the complex that is to say of the proteins TCP1, CCT2, CCT3, CCT4 and CCT5.

As used herein, the term “Dengue virus” as is general meaning in the art and denotes an RNA virus of the family Flaviviridae; genus Flavivirus. Other members of the same genus include yellow fever virus, West Nile virus, St. Louis encephalitis virus, Japanese encephalitis virus, tick-borne encephalitis virus, Kyasanur forest disease virus, and Omsk hemorrhagic fever virus. Most are transmitted by arthropods (mosquitoes or ticks), and are therefore also referred to as arboviruses (arthropod-borne viruses). According of the invention, The Dengue virus may be of any serotype, i.e. serotype 1, 2, 3 or 4.

As used herein, the “subject” denotes a mammal. Typically, a subject according to the invention refers to any subject (preferably human) afflicted with or susceptible to be afflicted with Dengue virus infection.

As used herein, the term “treatment” or “treat” refer to both prophylactic or preventive treatment as well as curative or disease modifying treatment, including treatment of subjects at risk of contracting the disease or suspected to have contracted the disease as well as subjects who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

Typically, the compound according to the invention includes but is not limited to a small organic molecule, an antibody, and a polypeptide like a mimetic or a variant.

In one embodiment, the compound according to the invention may be a low molecular weight compound, e. g. a small organic molecule (natural or not).

The term “small organic molecule” refers to a molecule (natural or not) of a size comparable to those organic molecules generally used in pharmaceuticals. The term excludes biological macromolecules (e. g., proteins, nucleic acids, etc.). Preferred small organic molecules range in size up to about 10000 Da, more preferably up to 5000 Da, more preferably up to 2000 Da and most preferably up to about 1000 Da.

In a particular embodiment, the inhibitor of the OST complex is the NGI-1 compound (see Lopez-Sambrooks et al 2016).

In one embodiment, the compound according to the invention is an antibody. Antibodies directed against the OST complex, the CCT complex or RACK1 can be raised according to known methods by administering the appropriate antigen or epitope to a host animal selected, e.g., from pigs, cows, horses, rabbits, goats, sheep, and mice, among others. Various adjuvants known in the art can be used to enhance antibody production. Although antibodies useful in practicing the invention can be polyclonal, monoclonal antibodies are preferred. Monoclonal antibodies against the OST complex, the CCT complex or RACK1 can be prepared and isolated using any technique that provides for the production of antibody molecules by continuous cell lines in culture. Techniques for production and isolation include but are not limited to the hybridoma technique originally described by Kohler and Milstein (1975); the human B-cell hybridoma technique (Cote et al., 1983); and the EBV-hybridoma technique (Cole et al. 1985). Alternatively, techniques described for the production of single chain antibodies (see e.g., U.S. Pat. No. 4,946,778) can be adapted to produce anti-GAS6 or anti-AXL single chain antibodies. Compounds useful in practicing the present invention also include anti-GAS6 or anti-AXL antibody fragments including but not limited to F(ab′)2 fragments, which can be generated by pepsin digestion of an intact antibody molecule, and Fab fragments, which can be generated by reducing the disulfide bridges of the F(ab′)2 fragments. Alternatively, Fab and/or scFv expression libraries can be constructed to allow rapid identification of fragments having the desired specificity to the OST complex, the CCT complex or RACK1.

Humanized anti-OST complex, anti-CTT complex or RACK1 antibodies and antibody fragments therefrom can also be prepared according to known techniques. “Humanized antibodies” are forms of non-human (e.g., rodent) chimeric antibodies that contain minimal sequence derived from non-human immunoglobulin. For the most part, humanized antibodies are human immunoglobulins (recipient antibody) in which residues from a hypervariable region (CDRs) of the recipient are replaced by residues from a hypervariable region of a non-human species (donor antibody) such as mouse, rat, rabbit or nonhuman primate having the desired specificity, affinity and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. Methods for making humanized antibodies are described, for example, by Winter (U.S. Pat. No. 5,225,539) and Boss (Celltech, U.S. Pat. No. 4,816,397).

Then, for this invention, neutralizing antibodies of the OST complex, the CCT complex or RACK1 are selected.

In one embodiment, the compound according to the invention is an anti-OST complex antibody like the ab121285 antibody of abcam (see for example www.abcam.com/OST-beta-antibody-ab121285.html).

In one embodiment, the compound according to the invention is an aptamer. Aptamers are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. and Gold L., 1990. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S. D., 1999. Peptide aptamers consists of a conformationally constrained antibody variable region displayed by a platform protein, such as E. coli Thioredoxin A that are selected from combinatorial libraries by two hybrid methods (Colas et al., 1996).

Then, for this invention, neutralizing aptamers of the OST complex, the CCT complex or RACK1 are selected.

In one embodiment, the compound according to the invention is a polypeptide.

In a particular embodiment the polypeptide is a functional equivalent, a variant or a mimetic of the proteins of the OST complex or of the proteins of the CTT complex or of RACK1. The term “functional equivalent” includes fragments, mutants, and muteins of the proteins. The term “functionally equivalent” thus includes any equivalent of the proteins obtained by altering the amino acid sequence, for example by one or more amino acid deletions, substitutions or additions such that the protein analogue retains the activity of the proteins. Amino acid substitutions may be made, for example, by point mutation of the DNA encoding the amino acid sequence.

Preferably, the functional equivalent is at least 80% homologous to the corresponding protein. In a particular embodiment, the functional equivalent is at least 90% homologous as assessed by any conventional analysis algorithm such as for example, the Pileup sequence analysis software (Program Manual for the Wisconsin Package, 1996).

The term “a functionally equivalent fragment” as used herein also may mean any fragment or assembly of fragments of the proteins of the proteins of the OST complex or proteins of the CTT complex or of RACK1. Accordingly the present invention provides a polypeptide capable of inhibiting the proteins of the OST complex or the proteins of the CTT complex or of RACK1.

The polypeptides of the invention may be produced by any suitable means, as will be apparent to those of skill in the art. Preferably, the polypeptide is produced by recombinant means, by expression from an encoding nucleic acid molecule. Systems for cloning and expression of a polypeptide in a variety of different host cells are well known.

When expressed in recombinant form, the polypeptide is preferably generated by expression from an encoding nucleic acid in a host cell. Any host cell may be used, depending upon the individual requirements of a particular system. Suitable host cells include bacteria mammalian cells, plant cells, yeast and baculovirus systems. Mammalian cell lines available in the art for expression of a heterologous polypeptide include Chinese hamster ovary cells. HeLa cells, baby hamster kidney cells and many others. Bacteria are also preferred hosts for the production of recombinant protein, due to the ease with which bacteria may be manipulated and grown. A common, preferred bacterial host is E coli.

In specific embodiments, it is contemplated that polypeptides used in the therapeutic methods of the present invention may be modified in order to improve their therapeutic efficacy. Such modification of therapeutic compounds may be used to decrease toxicity, increase circulatory time, or modify biodistribution. For example, the toxicity of potentially important therapeutic compounds can be decreased significantly by combination with a variety of drug carrier vehicles that modify biodistribution. In example adding dipeptides can improve the penetration of a circulating agent in the eye through the blood retinal barrier by using endogenous transporters.

A strategy for improving drug viability is the utilization of water-soluble polymers. Various water-soluble polymers have been shown to modify biodistribution, improve the mode of cellular uptake, change the permeability through physiological barriers; and modify the rate of clearance from the body. To achieve either a targeting or sustained-release effect, water-soluble polymers have been synthesized that contain drug moieties as terminal groups, as part of the backbone, or as pendent groups on the polymer chain.

Polyethylene glycol (PEG) has been widely used as a drug carrier, given its high degree of biocompatibility and ease of modification. Attachment to various drugs, proteins, and liposomes has been shown to improve residence time and decrease toxicity. PEG can be coupled to active agents through the hydroxyl groups at the ends of the chain and via other chemical methods; however, PEG itself is limited to at most two active agents per molecule. In a different approach, copolymers of PEG and amino acids were explored as novel biomaterials which would retain the biocompatibility properties of PEG, but which would have the added advantage of numerous attachment points per molecule (providing greater drug loading), and which could be synthetically designed to suit a variety of applications.

Those of skill in the art are aware of PEGylation techniques for the effective modification of drugs. For example, drug delivery polymers that consist of alternating polymers of PEG and tri-functional monomers such as lysine have been used by VectraMed (Plainsboro, N.J.). The PEG chains (typically 2000 daltons or less) are linked to the a- and e-amino groups of lysine through stable urethane linkages. Such copolymers retain the desirable properties of PEG, while providing reactive pendent groups (the carboxylic acid groups of lysine) at strictly controlled and predetermined intervals along the polymer chain. The reactive pendent groups can be used for derivatization, cross-linking, or conjugation with other molecules. These polymers are useful in producing stable, long-circulating pro-drugs by varying the molecular weight of the polymer, the molecular weight of the PEG segments, and the cleavable linkage between the drug and the polymer. The molecular weight of the PEG segments affects the spacing of the drug/linking group complex and the amount of drug per molecular weight of conjugate (smaller PEG segments provides greater drug loading). In general, increasing the overall molecular weight of the block co-polymer conjugate will increase the circulatory half-life of the conjugate. Nevertheless, the conjugate must either be readily degradable or have a molecular weight below the threshold-limiting glomular filtration (e.g., less than 60 kDa).

In addition, to the polymer backbone being important in maintaining circulatory half-life, and biodistribution, linkers may be used to maintain the therapeutic agent in a pro-drug form until released from the backbone polymer by a specific trigger, typically enzyme activity in the targeted tissue. For example, this type of tissue activated drug delivery is particularly useful where delivery to a specific site of biodistribution is required and the therapeutic agent is released at or near the site of pathology. Linking group libraries for use in activated drug delivery are known to those of skill in the art and may be based on enzyme kinetics, prevalence of active enzyme, and cleavage specificity of the selected disease-specific enzymes. Such linkers may be used in modifying the protein or fragment of the protein described herein for therapeutic delivery.

In another embodiment, the compound according to the invention is an inhibitor of the gene expression of the proteins of the OST complex or of the proteins of CTT complex or an inhibitor of the RACK1 gene expression.

Small inhibitory RNAs (siRNAs) can also function as inhibitors of the gene expression of the proteins of the OST complex or of the proteins of CTT complex or of RACK1 expression for use in the present invention. Gene expression of the proteins can be reduced by contacting a subject or cell with a small double stranded RNA (dsRNA), or a vector or construct causing the production of a small double stranded RNA (i.e. RNA interference or RNAi). Methods for selecting an appropriate dsRNA or dsRNA-encoding vector are well known in the art for genes whose sequence is known (e.g. see for example Tuschl, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and International Patent Publication Nos. WO 01/36646, WO 99/32619, and WO 01/68836).

Ribozymes can also function as inhibitors of the gene expression of the proteins of the OST complex or of the proteins of CTT complex or of the RACK1 gene expression for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of the proteins of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Both antisense oligonucleotides and ribozymes useful as inhibitors can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Antisense oligonucleotides siRNAs and ribozymes of the invention may be delivered in vivo alone or in association with a vector. In its broadest sense, a “vector” is any vehicle capable of facilitating the transfer of the antisense oligonucleotide siRNA or ribozyme nucleic acid to the cells and preferably cells expressing the proteins of interest. Preferably, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide siRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rouse sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

Preferred viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in Kriegler (1990) and in Murry (1991).

Preferred viruses for certain applications are the adeno-viruses and adeno-associated viruses, which are double-stranded DNA viruses that have already been approved for human use in gene therapy. The adeno-associated virus can be engineered to be replication deficient and is capable of infecting a wide range of cell types and species. It further has advantages such as, heat and lipid solvent stability; high transduction frequencies in cells of diverse lineages, including hemopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other vectors include plasmid vectors. Plasmid vectors have been extensively described in the art and are well known to those of skill in the art. See e.g. Sambrook et al., 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, eye, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

In a particular embodiment, the antisense oligonucleotide, siRNA, shRNA or ribozyme nucleic acid sequence is under the control of a heterologous regulatory region, e.g., a heterologous promoter. The promoter may be specific for Muller glial cells, microglia cells, endothelial cells, pericyte cells and astrocytes For example, a specific expression in Muller glial cells may be obtained through the promoter of the glutamine synthetase gene is suitable. The promoter can also be, e.g., a viral promoter, such as CMV promoter or any synthetic promoters.

In one embodiment, the inhibitors of the gene expression are directed to at least one gene coding for the proteins RPN1, DDOST, STT3A or STT3B.

Another object of the invention relates to a method for treating Dengue virus infection comprising administering to a subject in need thereof a therapeutically effective amount of an inhibitor of the OST complex and/or the CCT complex and/or RACK1.

In a one embodiment, said inhibitor is the compound NGI-1.

### Pharmaceutical Composition

Another object of the invention relates to a pharmaceutical composition comprising an effective dose of an inhibitor of the OST complex and/or of the CCT complex and/or of RACK1 for use in the treatment of Dengue virus infection in a subject in need thereof.

In another embodiment, the pharmaceutical composition may comprise at least one other antiviral compound as described below.

Any therapeutic agent of the invention may be combined with pharmaceutically acceptable excipients, and optionally sustained-release matrices, such as biodegradable polymers, to form therapeutic compositions.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

The form of the pharmaceutical compositions, the route of administration, the dosage and the regimen naturally depend upon the condition to be treated, the severity of the illness, the age, weight, and sex of the patient, etc.

The pharmaceutical compositions of the invention can be formulated for a topical, oral, intranasal, parenteral, intraocular, intravenous, intramuscular or subcutaneous administration and the like.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The doses used for the administration can be adapted as a function of various parameters, and in particular as a function of the mode of administration used, of the relevant pathology, or alternatively of the desired duration of treatment.

In addition, other pharmaceutically acceptable forms include, e.g. tablets or other solids for oral administration; time release capsules; and any other form currently can be used.

The pharmaceutical compositions according to the invention may be administered orally in the form of a suitable pharmaceutical unit dosage form. The pharmaceutical compositions of the invention may be prepared in many forms that include tablets, hard or soft gelatin capsules, aqueous solutions, suspensions, and liposomes and other slow-release formulations, such as shaped polymeric gels.

The mode of administration and dosage forms are closely related to the properties of the therapeutic agents or compositions which are desirable and efficacious for the given treatment application. Suitable dosage forms include, but are not limited to, oral, intravenous, rectal, sublingual, mucosal, nasal, ophthalmic, subcutaneous, intramuscular, transdermal, spinal, intrathecal, intra-articular, intra-arterial, sub-arachnoid, bronchial, and lymphatic administration, and other dosage forms for systemic delivery of active ingredients.

Pharmaceutical compositions of the invention may be administered by any method known in the art, including, without limitation, transdermal (passive via patch, gel, cream, ointment or iontophoretic); intravenous (bolus, infusion); subcutaneous (infusion, depot); transmucosal (buccal and sublingual, e.g., orodispersible tablets, wafers, film, and effervescent formulations; conjunctival (eyedrops); rectal (suppository, enema)); or intradermal (bolus, infusion, depot).

Oral liquid pharmaceutical compositions may be in the form of, for example, aqueous or oily suspensions, solutions, emulsions, syrups or elixirs, or may be presented as a dry product for constitution with water or other suitable vehicle before use. Such liquid pharmaceutical compositions may contain conventional additives such as suspending agents, emulsifying agents, non-aqueous vehicles (which may include edible oils), or preservatives.

Pharmaceutical compositions of the invention may also be formulated for parenteral administration (e.g., by injection, for example, bolus injection or continuous infusion) and may be presented in unit dosage form in ampoules, pre-filled syringes, small volume infusion containers or multi-dose containers with an added preservative. The pharmaceutical compositions may take such forms as suspensions, solutions, or emulsions in oily or aqueous vehicles, and may contain formulating agents such as suspending, stabilizing and/or dispersing agents. Alternatively, the pharmaceutical compositions of the invention may be in powder form, obtained by aseptic isolation of sterile solid or by lyophilization from solution, for constitution with a suitable vehicle, e.g. sterile, pyrogen-free water, before use.

Pharmaceutical compositions suitable for rectal administration wherein the carrier is a solid are most preferably presented as unit dose suppositories. Suitable carriers include cocoa butter and other materials commonly used in the art, and the suppositories may be conveniently formed by admixture of the pharmaceutical composition with the softened or melted carrier(s) followed by chilling and shaping in molds.

For administration by inhalation, the pharmaceutical compositions according to the invention are conveniently delivered from an insufflator, nebulizer or a pressurized pack or other convenient means of delivering an aerosol spray. Pressurized packs may comprise suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the pharmaceutical compositions of the invention may take the form of a dry powder composition, for example, a powder mix of the pharmaceutical composition and a suitable powder base such as lactose or starch. The powder composition may be presented in unit dosage form in, for example, capsules or cartridges or, e.g., gelatin or blister packs from which the powder may be administered with the aid of an inhalator or insufflator.

For intra-nasal administration, the pharmaceutical compositions of the invention may be administered via a liquid spray, such as via a plastic bottle atomizer. Typical of these are the Mistometerg (isoproterenol inhaler-Wintrop) and the Medihaler® (isoproterenol inhaler-Riker).

For antisense nucleic acid administration, the pharmaceutical compositions of the 25 invention may be prepared in forms that include encapsulation in liposomes, microparticles, microcapsules, lipid-based carrier systems. Non limiting examples of alternative lipid based carrier systems suitable for use in the present invention include polycationic polymer nucleic acid complexes (see, e.g. US Patent Publication No 20050222064), cyclodextrin polymer nucleic acid complexes (see, e.g. US Patent Publication No 20040087024), biodegradable poly 3 amino ester polymer nucleic acid complexes (see, e.g. US Patent Publication No 20040071654), pH sensitive liposomes (see, e.g. US Patent Publication No 20020192274), anionic liposomes (see, e.g. US Patent Publication No 20030026831), cationic liposomes (see, e.g. US Patent Publication No 20030229040), reversibly masked lipoplexes (see, e.g. US Patent Publication No 35 20030180950), cell type specific liposomes (see, e.g. US Patent Publication No 20030198664), microparticles containing polymeric matrices (see, e.g. US Patent Publication No 20040142475), pH sensitive lipoplexes (see, e.g. US Patent Publication No 20020192275), liposomes containing lipids derivatized with releasable hydrophilic polymers (see, e.g. US Patent Publication No 20030031704), lipid en trapped nucleic acid (see, e.g. PCT Patent Publication No WO 03/057190), lipid encapsulated nucleic acid (see, e.g. US Patent Publication No 20030129221), polycationic sterol 5 derivative nucleic acid complexes (see, e.g. U.S. Pat. No. 6,756,054), other liposomal compositions (see, e.g. US Patent Publication No 20030035829), other microparticle compositions (see, e.g. US Patent Publication No 20030157030), poly-plexes (see, e.g. PCT Patent Publication No WO 03/066069), emulsion compositions (see, e.g. U.S. Pat. No. 6,747,014), condensed nucleic acid complexes (see, e.g. US Patent Publication No 20050123600), other polycationic nucleic acid complexes (see, e.g. US Patent Publication No 20030125281), polyvinylether nucleic acid complexes (see, e.g. US Patent Publication No 20040156909), polycyclic amidinium nucleic acid complexes (see, e.g. US Patent Publication No 20030220289), nanocapsule and microcapsule compositions (see, e.g. PCT Patent 15 Publication No WO 02/096551), stabilized mixtures of liposomes and emulsions (see, e.g. EP1304160), porphyrin nucleic acid complexes (see, e.g. U.S. Pat. No. 6,620,805), lipid nucleic acid complexes (see, e.g. US Patent Publication No 20030203865), nucleic acid micro emulsions (see, e.g. US Patent Publication No 20050037086), and cationic lipid based compositions (see, e.g. US Patent Publication No 20050234232). One skilled in the art will appreciate that modified siRNA of the present invention can also be delivered as a naked siRNA molecule.

Pharmaceutical compositions of the invention may also contain other adjuvants such as flavorings, colorings, anti-microbial agents, or preservatives.

It will be further appreciated that the amount of the pharmaceutical compositions required for use in treatment will vary not only with the therapeutic agent selected but also with the route of administration, the nature of the condition being treated and the age and condition of the patient and will be ultimately at the discretion of the attendant physician or clinician.

The administration regimen may be a systemic regimen. The mode of administration and dosage forms are closely related to the properties of the therapeutic agents or compositions which are desirable and efficacious for the given treatment application. Suitable dosage forms and routes of administration include, but are not limited to, oral, intravenous, rectal, sublingual, mucosal, nasal, ophthalmic, subcutaneous, intramuscular, transdermal, spinal, intrathecal, intra-articular, intra-arterial, sub-arachnoid, bronchial, and lymphatic administration, and/or other dosage forms and routes of administration for systemic delivery of active ingredients. In a preferred embodiment, the dosage forms are for parenteral administration.

The administration regimen may be for instance for a period of at least 5, 6, 7, 8, 9, 10, 15, 20, 25, 30, 35, 40, 45, 50, 55, 60, 65, 70, 75, 80, 85, 90, 95, 100 days.

The dose range may be between 0.1 mg/kg/day and 100 mg/kg/day. More preferably, the dose range is between 0.5 mg/kg/day and 100 mg/kg/day. Most preferably, the dose range is between 1 mg/kg/day and 80 mg/kg/day. Most preferably, the dose range is between 5 mg/kg/day and 50 mg/kg/day, or between 10 mg/kg/day and 40 mg/kg/day.

In some embodiments, the dose may be of at least 0.1, 0.2, 0.3, 0.4, 0.5, 0.6, 0.7, 0.8, 0.9, 1, 1.5, 2, 2.5, 3, 3.5, 4, 4.5, 5, 6, 7, 8, 9, 10, 15, 20, 25, 30, 35, 40, 45, 50 10 mg/kg/day. In some embodiments, the dose may be of at most 50, 45, 40, 35, 30, 25, 20, 25, 15, 10, 5, 1, 0.5, 0.1 mg/kg/day.

The dose range may also be between 10 to 10000 UI/kg/day. More preferably, the dose range is between 50 to 5000 UI/kg/day, or between 100 to 1000 UI/kg/day.

In some embodiments, the dose may be of at least 10, 25, 50, 75, 100, 150, 200, 15 250, 300, 350, 400, 450, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10000 UI/kg/day. In some embodiments, the dose may be of at most 10000, 9500, 9000, 8500, 8000, 7500, 7000, 6500, 6000, 5500, 5000, 4500, 4000, 3500, 3000, 2500, 2000, 1500, 1000, 900, 800, 600, 500, 450, 400, 350, 300, 250, 200, 150, 100 UI/kg/day.

### Antiviral Compounds

In another aspect, the compounds according to the invention are for administration in combination with at least one other antiviral compound, either sequentially or simultaneously.

In one embodiment, the invention also relates to i) a compound according to the invention, and ii) another antiviral compound, as a combined preparation for simultaneous, separate or sequential for use in the treatment of Dengue virus infection in a subject in need thereof.

Sequential administration indicates that the components are administered at different times or time points, which may nonetheless be overlapping. Simultaneous administration indicates that the components are administered at the same time.

The antiviral compound may include, but is not limited to, neuraminidase inhibitors, viral fusion inhibitors, protease inhibitors, DNA polymerase inhibitors, signal transduction inhibitors, reverse transcriptase inhibitors, interferons, nucleoside analogs, integrase inhibitors, thymidine kinase inhibitors, viral sugar or glycoprotein synthesis inhibitors, viral structural protein synthesis inhibitors, viral attachment and adsorption inhibitors, viral entry inhibitors and their functional analogs.

Neuraminidase inhibitors may include oseltamivir, zanamivir and peramivir. Viral fusion inhibitors may include cyclosporine, maraviroc, enfuviritide and docosanol.

Protease inhibitors may include saquinavir, indinarvir, amprenavir, nelfinavir, ritonavir, tipranavir, atazanavir, darunavir, zanamivir and oseltamivir.

DNA polymerase inhibitors may include idoxuridine, vidarabine, phosphonoacetic acid, trifluridine, acyclovir, forscarnet, ganciclovir, penciclovir, cidoclovir, famciclovir, valaciclovir and valganciclovir.

Signal transduction inhibitors include resveratrol and ribavirin. Nucleoside reverse transcriptase inhibitors (NRTIs) may include zidovudine (ZDV, AZT), lamivudine (3TC), stavudine (d4T), zalcitabine (ddC), didanosine (2′,3′-dideoxyinosine, ddI), abacavir (ABC), emirivine (FTC), tenofovir (TDF), delaviradine (DLV), fuzeon (T-20), indinavir (IDV), lopinavir (LPV), atazanavir, combivir (ZDV/3TC), kaletra (RTV/LPV), adefovir dipivoxil and trizivir (ZDV/3TC/ABC). Non-nucleoside reverse transcriptase inhibitors (NNRTIs) may include nevirapine, delavirdine, UC-781 (thiocarboxanilide), pyridinones, TIBO, calanolide A, capravirine and efavirenz.

Viral entry inhibitors may include Fuzeon (T-20), NB-2, NB-64, T-649, T-1249, SCH-C, SCH-D, PRO 140, TAK 779, TAK-220, RANTES analogs, AK602, UK-427, 857, monoclonal antibodies against relevant receptors, cyanovirin-N, clyclodextrins, carregeenans, sulfated or sulfonated polymers, mandelic acid condensation polymers, AMD-3100, and functional analogs thereof.

The invention will be further illustrated by the following figures and examples. However, these examples and figures should not be interpreted in any way as limiting the scope of the present invention.

### FIGURES

FIG. 1. RACK1, CCT and OST complexes are required for DENY replication. (A) HeLa cells were transfected with siRNA pool targeting a representative panel of HDFs. Two days post transfection cells were challenged with DENV (MOI 2). At 48 hpi, level of infection and virus titer were determined and normalized to non-targeting siRNA transfected cells. Data shown are means±SD of three independent experiments. (B) DENV2-replicon cells were reverse transfected by the indicated siRNAs pool. At 72h post-transfection, GFP signal reflecting DENV RNA replication was quantified by flow cytometry. Data shown are means±SD of three independent experiments. Data shown are means±SD of three independent experiments. Significance was calculated using a one-way ANOVA statistical test with a Dunnett's multiple comparison test. (n.s., not significant;*p<0.01; **p<0.001; ***p<0.0001).

FIG. 2. Catalytic activity of the OST complex is essential for DENY replication. Silencing of different OST subunits affects DENV and ZIKV NS1 glycosylation. (A) HeLa cells were pre-incubated for 24h with increased concentration of NGI-1, or for 3h with MPA prior infection with DENV2_16681 (MOI 5). MPA, Mycophenolic acid. (B) Replication kinetics of DENV2 in HeLa replicon cells challenged with NGI-1 (2 μM) or MPA (10 μM) along the experiment duration. (C) Supernatants from HEK-293T cells transfected with FLAG-NS plasmids, were subjected to ELISA to quantify secreted NS1. (D) HeLa cells were incubated for 24 hours with NGI-1 and challenged in continuous presence of the drug with DENV1 (MOI 1), DENV2_16681 (MOI 5), DENV2_JAM (MOI 5), DENV3_THAI (MOI 5), WNV (MOI 1), ZIKV (MOI 1), CHIKV (MOI 5) or HSV (MOI 0.3). (E) Similarly treated primary skin fibroblast and monocyte derived dendritic cells (MDDC) were challenged with DENV2_16681 (MOI 5) and ZIKV_HD78788 (MOI 2). (D and E) The levels of infected cells were assessed 24h post infection by flow cytometry using respective anti-viral antibodies. (A-E) Data shown are means±SD of two independent experiments. Significance was calculated using a one-way ANOVA statistical test with a Dunnett's multiple (A, B and C) or Tukey comparison test (D and E). (n.s., not significant; **p<0.001; ***p<0.000).

FIG. 3. Cell proliferation is not affected by NGI-1. Cell proliferation is measured over time after incubation with NGI-1 (2 μM), MPA (10 μM) or respective mock control. Data shown are means±S.D. of one representative experiment of three replicates. RLU, relative light units.

### EXAMPLE

Material & Methods

Cells.

Human microglia CHME3, human embryonic kidney 293T, A549, HeLa and Vero cells were maintained in Dulbecco's modified Eagle's medium (DMEM; Invitrogen Life Technologies) supplemented with 10% heat-inactivated fetal bovine serum (FBS) and 1% penicillin/streptomycin (P/S). Raji cells were maintained in RPMI medium 1640 (Invitrogen Life Technologies) supplemented with 10% FBS and 1% (P/S). HAP1 were purchased from Horizon Genomics and maintained according to the manufactured conditions. The AP61 mosquito cells (National Reference Centre for Arboviruses, Pasteur Institute, Paris) were maintained as previously described (Meertens et al., 2012).

Plasmids.

The FLAG-HA-tagged Replicon was generated as follow. The restriction fragments FseI-SalI of pDENV2-rep-GZ vector (Ansarah-Sobrinho et al., 2008) was used as template in overlap extension PCRs to insert the FLAG-HA-epitope at the N-terminal of NS1 (see primer table 51). PCR product was cloned back into the pDENV2-rep-GZ by using InfusionHD cloning (Takara Bio). The firefly luciferase (Fluc)-expressing reporter constructs were described previously (Edgil et al., 2006). FLAG-HA-tagged NS1 and NS4B proteins were amplified by PCR from the pDENV2-rep-GZ vector, and cloned in EcoRI-BamHI (NS5, NS4A or NS4B) digested pLVX-IRES-ZsGreenlvector (Takara). Expressing vectors of glycosylation mutants of DENV2 NS1 (N130Q, N207Q, and N130Q/N207Q) were generated using the Quick Change Site Directed Mutagenesis Kit (Agilent).

Virus Preparation and Titration.

Viruses were prepared and titrated as previously described (Meertens et al., 2012). Viral titers were determined on Vero cells by flow cytometry analysis and expressed as Flow cytometry Infectious Units (FIU). The DENV2 Rluc reporter virus (DENV-R2A) was produced as previously described (Fischl and Bartenschlager, 2013) and infection was determined by measuring the Rluc activity using TriStar² LB942 microplate reader (Berthold Technologies).

Two-Step Immunoprecipitation Procedure.

Two-step immunoprecipitation was performed as previously described (Nakatani and Ogryzko, 2003). Briefly, 5.108 HeLa, Raji, or Hap1 cells, expressing either the WT or the Flag-HA-tagged DENV2 replicon, were lysed for 30 min in cold IP Lysis Buffer (Invitrogen cat #87788) supplemented with complete protease and phosphatase inhibitors (Roche Diagnostics, Basel, Switzerland), and then cleared by centrifugation for 30 min at 6,000 g. Supernatants were incubated overnight at 4° C., with anti-Flag magnetic beads (Sigma # M8823). Beads were washed three times with B015 buffer (20 mMTris-HCl pH 7.4, 150 mM NaCl, 5 mM MgCl2, 10% Glycerol, 0.5 mM EDTA, 0.05% Triton, 0.1% Tween-20), and immune complexes were eluted twice with 3×Flag-peptide (200 μg/ml; SIGMA F4799-4MG) for 30 min at room temperature (RT). FLAG-IP complexes were further incubated with HA magnetic beads (Pierce #88837) for 6 h at 4° C., washed 3 times with B015, and immune complexes were eluted twice with HA peptide (400 μg/ml; Roche #11666975001). Eluates were concentrated on a Pierce Concentrator, PES, 10K (Pierce #88513) and stored at −20° C. until use. Samples were analyzed by mass spectrometry at the Taplin Biological Mass Spectrometry facility (Harvard Medical School, Boston, Mass.).

Network Analysis.

A total of five co-affinity purifications and MS analysis experiments were performed with the NS1 tagged DENV-2 replicon in Raji (Raji-1 and 2), HeLa (HeLa-1 and 2) and HAP1 cells. We selected a set of 277 host proteins that co-purified in all three cell lines for further analysis. DAVID 6.8 was used to identify statistical enrichments for specific GO terms from the “Cellular Component” (CC) and “Biological Process” (BP) categories (Huang et al., 2009a, 2009b). The corresponding interaction network was built using Cytoscape 3.4.0 (Shannon et al., 2003), and proteins were clustered into functional modules using enriched GO terms as a guideline and manual mining of literature. Besides, known physical interactions between HRFs or HDFs were retrieved using the GeneMANIA server (Warde-Farley et al., 2010), and corresponding networks were displayed using Cytoscape. We also used GeneMANIA to identify, among cellular proteins that co-purified with DENV replicon in at least two different cell types, some direct binding partners of HRFs or HDFs. Those showing some impact on DENV2 infection when tested by siRNA.

Mass Spectrometry Analysis.

A total of five co-affinity purifications and MS analysis experiments were performed with the NS1 tagged DENV2 replicon or the untagged replicon as a control in Raji (IP-1 and 2), HeLa (IP-3 and 4) and HAP1 (IP-5) cells. Samples were analyzed at Taplin Biological Mass Spectrometry Facility (Harvard Medical School). Briefly, concentrated eluates issued from Immunopurification of WT and FLAG-HA-tagged replicon are separated on 10% Tris-glycine SDS-PAGE gels (Invitogen), and stained with Imperial™ Protein Stain (Thermo Fisher). Individual regions of the gel were cut into approximately 1 mm3 pieces and subjected to a modified in-gel trypsin digestion procedure (Shevchenko et al., 1996). Peptides were desalted and subjected to a nano-scale reverse-phase HPLC (Peng and Gygi, 2001). Eluted peptides are then subjected to electrospray ionization and then MS/MS analysis into an LTQ Orbitrap Velos Pro ion-trap mass spectrometer (Thermo Fisher Scientific, Waltham, Mass.). Peptides were detected, isolated, and fragmented to produce a tandem mass spectrum of specific fragment ions for each peptide. Peptide sequences were determined by matching protein databases with the acquired fragmentation pattern by the Sequest software program (Thermo Fisher Scientific, Waltham, Mass.) (Eng et al., 1994). All databases include a reversed version of all the sequences and the data was filtered to ≤2% peptide false discovery rate.

Small Interfering RNA (siRNA) Screen Assay.

The proteins identified by our AP-MS approach and meeting our selection threshold, were targeted by an ON-TARGETplus SMARTpool siRNA library (Dharmacon). HeLa cells were transfected using the Lipofectamine RNAiMax protocol (Life Technologies) with 30 nM final siRNA following manufacturer's protocol. 48 hours post-transfection, cells were infected with DENV2_16681 at MOI 5 for 48h, and infection was quantified by flow cytometry using the anti-prM 2H2 antibody (data not shown). Supernatants were used to infect naïve VeroE6 cells for 24h for viral titration, which is representative of the infectious particles release (data not shown). The screen was performed in high-throughput format, and the data shown represent three independent experiments. Three siRNA controls were included in the screen: 1) A non-targeting siRNA used as a reference, 2) A siATP6V1B2 targeting the ATP6V1B2 gene expression, which impairs flavivirus pH-dependent fusion and vRNA release (Fernandez-Garcia et al., 2011), which serves as a positive control for host dependency factors (HDFs). 3) Finally, a siIFIT1 for the knock down of IFIT1, a well documented ISG known to inhibit viral replication, which serves as positive control for host restriction factors (HRFs) (Fensterl and Sen, 2015). Cutoffs are <50% for HDFs and >200% for HRFs as compared to siNT.

Statistical Analyses.

Graphical representation and statistical analyses were performed using Prism5 software (GraphPad Software). Unless otherwise stated, results are shown as means+/−standard deviation (SD) from three independent experiments. Differences were tested for statistical significance using the unpaired two-tailed t test or One-way Anova with Tukey post-test.

Results

Mapping the NS1 Interactome During DENV Replication

Replicons are self-replicating flavivirus RNAs containing large in-frame deletions in the structural genes and are useful tools to study translation and RNA amplification of several flaviviruses (Mukherjee et al., 2014). To establish a global map of NS1-host protein interactions occurring during DENV replication, we stably expressed a DENV2 replicon encoding NS1 tagged with N-terminal FLAG and HA epitopes (FH-NS1) or the untagged version (Wt) in three different human cell lines (Raji, HeLa and HAP-1) (data not shown). We first confirmed that cells expressing the Wt or FH-NS1 replicons showed comparable replication levels (as determined by viral RNA quantification), and induced membrane rearrangements similar to those observed in DENV-infected cells (data not shown). The FH-tag did not affect DENV NS protein expression (data not shown) nor NS1 secretion (data not shown). Following tandem affinity of FH-NS1 (data not shown), co-immunoprecipitated proteins were separated by SDS-PAGE gel electrophoresis, visualized by silver-staining and subjected to mass spectrometry (MS) analysis (data not shown). All the NS proteins were efficiently co-purified with NS1 at various ratios, as indicated by the total peptide count relative to each protein (data not shown). The peptide distribution throughout the viral polyprotein sequence and the protein coverage were relatively comparable between the three cells lines tested (data not shown). Glycerol gradient fractionation of the affinity-purified material followed by western-blotting shows that NS1, NS3 and NS4B are found in the same fractions, strongly suggesting that they are part of the same complex (data not shown). We additionally identified 742, 1023, and 773 host factors that co-purified with the FH-NS1 in Raji, HeLa, and HAP1 cells, respectively (data not shown). To strengthen our results, all subsequent analyses were limited to the list of 277 host proteins that overlapped between the three cells lines (data not shown). We also highlighted statistical enrichment for specific biological processes (BP) or cellular components (CC) as determined by Gene Ontology (GO) analysis data not shown). The 277 host proteins clustered into 16 groups representing prominent cellular modules interacting with the NS1-tagged replicon (data not shown). The NS1 interactome is significantly enriched in mitochondrial proteins such as VDAC1, VDAC2, AIFM1, TIMM50 and the recently identified mitophagic receptor PHB2 (Wei et al., 2017). In addition, the autophagic SH3GLB1 and SH3GLB2 proteins, two members of the endophilin family that possess membrane binding and liposome tubulation activities (Mim and Unger, 2012), were identified in our study, suggesting some contribution to membrane curvature, autophagosome formation and/or other membrane remodeling induced during DENV infection. We also found multiple components of the Golgi apparatus and the endoplasmic reticulum (ER) including components of the ER-associated protein degradation pathway (SEL1L, AUP1, HM13) and the oligosaccharyltransferase complex (OST). The cytosolic chaperonin-containing T (CCT) complex including TCP1, CCT2, CCT3 and CCT5 was also significantly enriched, suggesting that these proteins may be involved in the chaperoning and proper folding of viral factors. Strikingly, several nuclear proteins were also highly represented such as components of the nuclear pore (XPO5, TNPO1) and the machineries of DNA repair and replication (FANCI, PCNA, MSH2 and MCM3) that could be linked to activation of the DNA damage pathway induced during DENV infection and immune responses against the virus.

Identification of DENV Host Restriction and Dependency Factors

To further pinpoint the function of the 277 selected host factors interacting with the NS1-tagged replicon, we silenced their expression by RNA interference and determined consequences on virus infection (data not shown). Our study identified 34 host restriction factors (HRFs) whose silencing enhanced viral infection by 2-4 fold (data not shown) as well as 56 host dependency factors (HDFs) whose silencing decreased viral infection by 10-100 fold (data not shown). The list was extended by literature mining for protein-protein interaction involving the identified HRF and HDF (data not shown). Endogenous DDOST, SPCS2, CCT2, CCT3, CCT5, RACK1, NOMO1, MCM3, MSH2 and PHB2 were immunoprecipitated with the FH-NS1 replicon, confirming that they are true DENV NS partners (data not shown).

HRFs were globally distributed among the different cellular modules (data not shown) including autophagy proteins (EI24, ATG9A, SCAMP3), key components of the gamma secretase complex (NCSTN), the Nodal signaling pathway (NOMO1, NCLN) and mitochondrial factors such PHB2, TIMM50, AIFM1 or AGK. Interestingly, proteins involved in the DNA damage response (MSH2, MCM3) and two E3 Ubiquitin ligases that regulate this pathway (UBRS and its paralog HUWE1) were also found to restrict DENV infection. We also identified several factors involved in mRNA translation and innate immunity such as EIF2AK2 (PKR), the aminoacyl-tRNA synthetase EPRS and its binding partner PCBP2 that were previously characterized as negative modulators of MAVS signaling and innate immunity (Lee et al., 2016). Furthermore, BZW1 and BZW2, two proteins known to negatively control mRNA translation (Kozel et al., 2016) also inhibited viral growth. Validation experiments showed that NOMO1, MCM3, and PHB2 knockdown resulted in significant enhancement of infectious virion production (data not shown).

Among the HDFs, we found several factors involved in polypeptide-associated translation and processing (ECM1, SPCS2, SEC61A1 and SEC63), as well as subunits of the OST complex (STT3A, STT3B, DDOST, RPN1 and RPN2) (data not shown). We also identified molecules involved in protein folding such as the CCT complex (TCP1, CCT2, CCT3, CCT4, CCT5), RNA translation (RACK1, RPS25, EEF2), Golgi-associated vesicular transport (COPG1, ARF1, GBF1, NSF, SCFD1), the ERAD pathway (SEL1L, HM13, FAF2, and HSP90B1), and nuclear function (PCNA, CSE1L, NUP210) (data not shown).

RACK1, CCT and OST Complexes are Required for DENV Replication

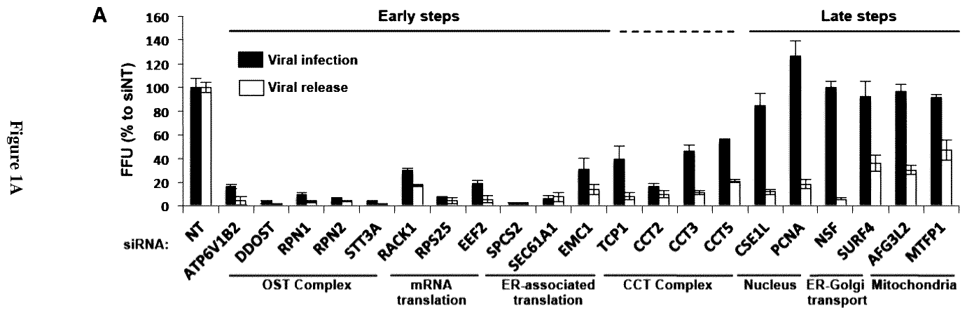

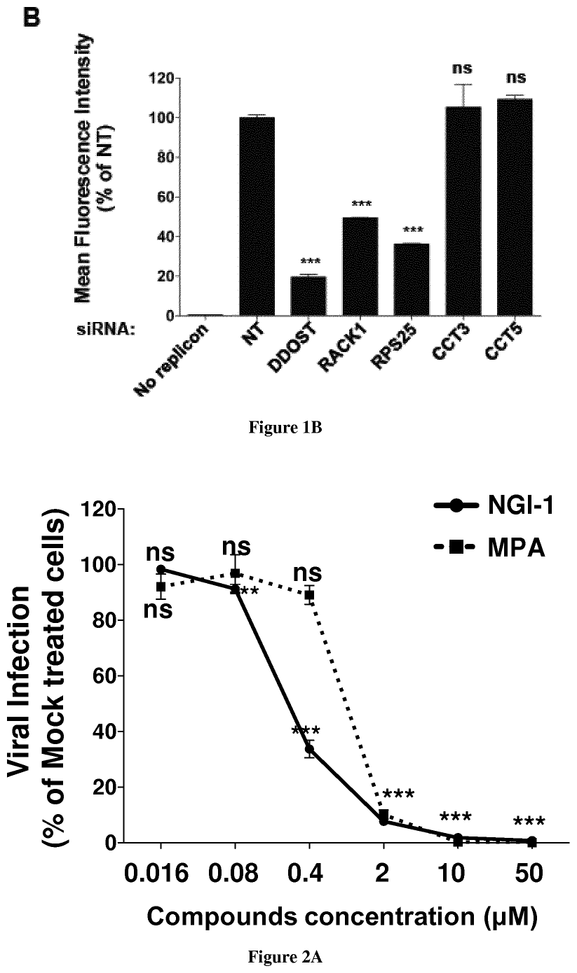

Since our goal was to identify potential targets for antiviral strategies, we next focused on the HDFs found in our screen. To dissect the viral steps where these host factors are required, we knocked down the expression of selected HDFs representative of each module in HeLa cells and determined their impact on the de novo prM protein synthesis (viral infection) and the release of infectious particles present in the supernatant of infected cells (viral release) (FIG. 1A). The majority of the HDFs impacted the early steps of the DENV life cycle (viral infection, FIG. 1A) including proteins involved in N-linked glycosylation (DDOST, STT3A, RPN1 and RPN2), RNA translation (EEF2, RPS25 and RACK1), peptide translocation and processing (SEC61A1, SPCS2, ECM1), and protein folding (TCP1, CCT2, 3 and 5). Silencing other protein such as CSE1L, PCNA, NSF, SURF4, AFG3L1, and MTFP1 decreased the release of infectious particles without impacting the prM synthesis, indicating that these factors are involved in the late stages of the DENV life cycle. Data presented in FIG. 1A were confirmed using a DENV2 RLuc reporter virus (Fischl and Bartenschlager, 2013) which allows investigating DENV replication (data not shown). We then focused our study on the major hits of our screen: RPS25, RACK1, the CCT and OST complexes. Using the DENV replicon system that bypasses viral entry, we show that depletion of DDOST, RACK1 or RPS25 strongly decreased GFP reporter expression (FIG. 1B), indicating an essential role on these molecules on vRNA amplification. However, CCT3 or CCT5 silencing had no effect of DENV RNA replication (FIG. 1B) and excerts a marginal role in virus entry (data not shown). Despite close interactions with DENV NS proteins, the CCT complex could probably acts on structural proteins c-prM-E, which are not present in our replicon system. Consistently, glycerol gradient sedimentation experiments on proteins co-purified with FH-tagged NS1 confirmed that RACK1, DDOST and NS3 (a major constituent of the DENV RC) have a near-identical distribution ranging from fraction 12 to 16, indicating that they are part of the same complex (data not shown). In contrast CCT3 and CCT5 peaked at fractions 18 and 20 and were excluded from the NS3 containing fractions. Moreover, in order to discriminate between vRNA translation and amplification, RACK1, RPS25, and DDOST depleted HeLa cells were challenged with a DENV2 RLuc virus to monitor the kinetic of viral infection (data not shown). A small peak of the Luc activity was detected at 4h post infection reflecting the initial translation of the incoming vRNA by the cellular machinery. This is followed by a marked increase of the Luc activity as a consequence of vRNA amplification (data not shown). The inhibition of RACK1, RPS25 or DDOST had no impact on the initial vRNA translation step but strongly impaired DENV RLuc replication (data not shown). As a positive control, knockdown of the ATP6V1B2 gene, which impairs flavivirus pH-dependent fusion and vRNA release (Fernandez-Garcia et al., 2011), strongly inhibited the initial RNA translation step. Similar results were obtained when vRNA was transfected into the cells to bypass viral entry (data not shown). In agreement with these observations, depletion of DDOST, RACK1 or RPS25 had no impact on the expression of translation reporter construct containing the firefly Luc gene flanked with either the DENV2 5′ UTR and 3′ UTR sequences or the human β-globin (βg) 5′ UTR and a 60-mer poly(A) tail, respectively (Edgil et al., 2006) (data not shown). Together these results establish an important role for RACK1, RPS25 and DDOST in vRNA amplification but not in the initial translation step. DENV dependency on RACK1 and DDOST was confirmed in two additional human cell lines, CHEM3 and A549 (data not shown). RACK1 and DDOST were also required for infection of HeLa cells by ZIKV and West Nile Virus (WNV) but not Herpes Simplex Virus-1 (HSV-1) or HIV pseudoparticles bearing the vesicular stomatitis virus G protein (VSVpp) (data not shown). Overall these data showed that RACK1, CCT and OST complexes are required for DENV replication and play distinct roles during the viral life cycle.

OST Complex Catalytic Activity is Essential for DENV Replication

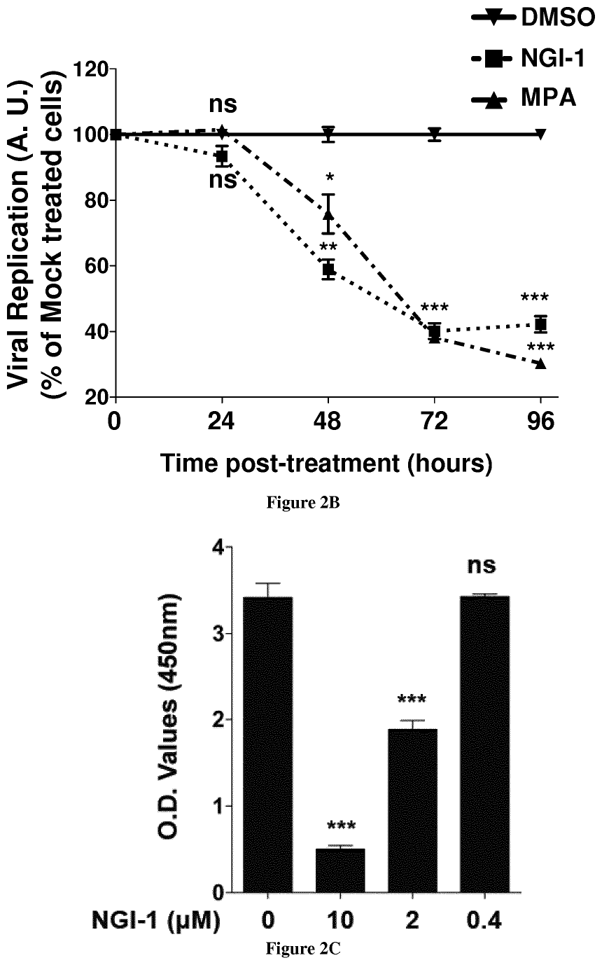

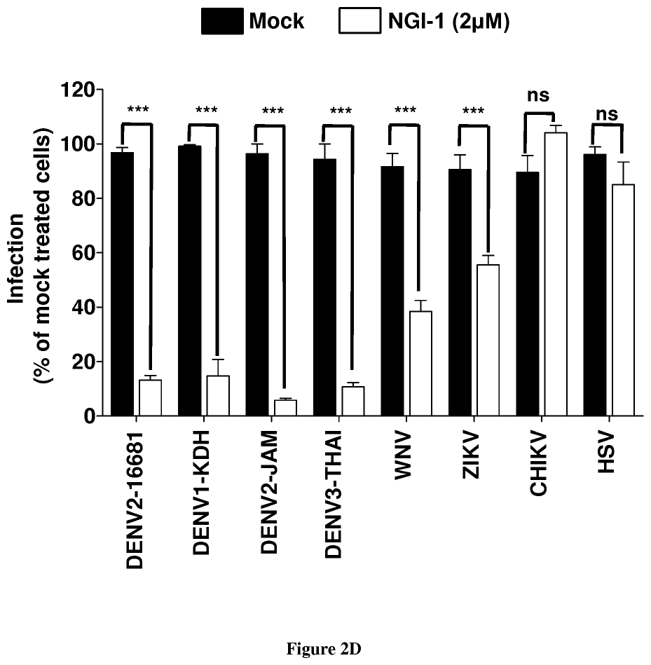

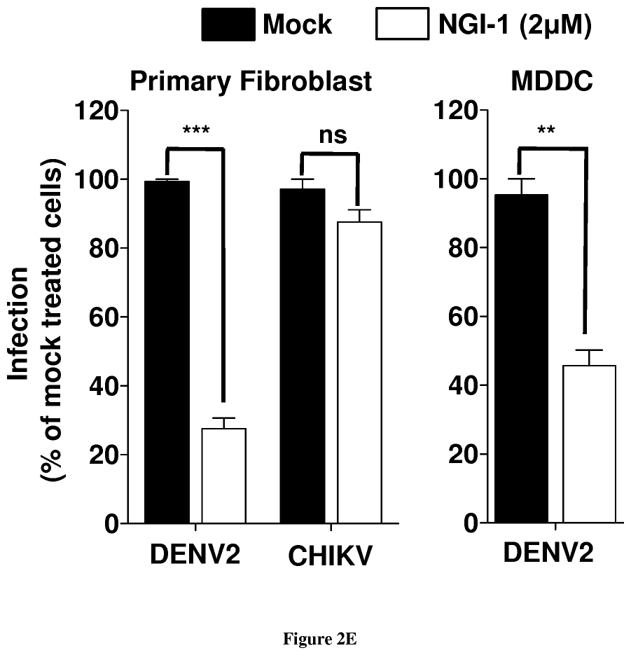

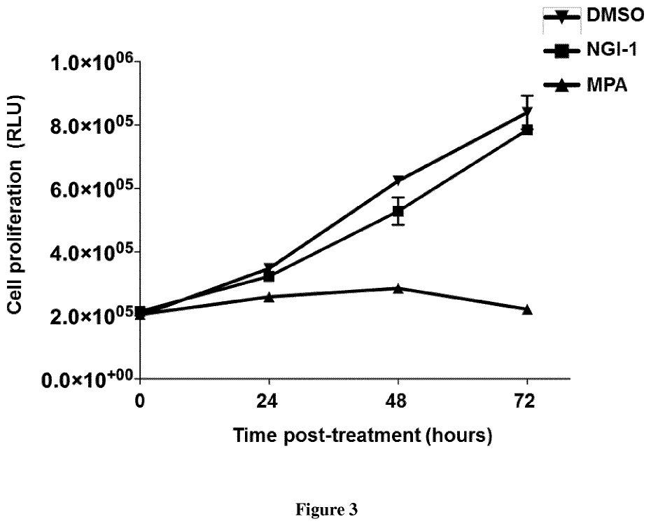

The OST complex is known to be required for flavivirus infection through mechanisms that are poorly understood (Marceau et al., 2016; Savidis et al., 2016; Zhang et al., 2016). In mammalians, there are two OST complexes, each composed of a catalytic subunit isoform (STT3A or STT3B) associated with a set of shared subunits (RPN1, RPN2, DDOST, DAD1 and OST4) (Cherepanova et al., 2016). Their main function is to transfer a preassembled oligosaccharide to selected asparagine residues within the consensus sequence asparagine-X-serine/threonine (Cherepanova et al., 2016). We speculate that the OST complex may regulate DENV NS protein glycosylation and function. Consistent with previous studies (Naik and Wu, 2015; Pryor and Wright, 1994; Somnuke et al., 2011), we observed that NS1 and NS4B and not the other NS were N-glycosylated (data not shown). Pull-down experiments confirmed that the endogenous OST complex, exemplified here with DDOST, interacts with NS1 or NS4B ectopically expressed in HEK-293T cells (data not shown). DDOST interacts also with NS3, which is consistent with a recent study (Marceau et al., 2016). To test whether the OST complex contributes to NS1 or NS4B N-glycosylation, we transfected HEK-293T cells with a siRNA pool targeting RPN1, DDOST, STT3A or STT3B. Because the STT3B complex can glycosylate sites that are skipped by the STT3A complex (Cherepanova et al., 2016; Ruiz-Canada et al., 2009), cells were also co-transfected with siRNA targeting both isoforms (STT3A+STT3B) (data not shown). Upon co-expression of DENV FLAG-NS1, lower bands corresponding to non-glycosylated NS1 clearly appeared in DDOST, RPN1 or STT3A/3B-depleted cells, but not in cells silenced for each catalytic subunit individually (data not shown). Similar results were obtained when HEK-293T cells were transfected with ZIKV NS1 (data not shown) or DENV NS4B (data not shown) while silencing OST components showed no effect on NS3 migration (data not shown). NS1 secretion was significantly impaired in cells depleted for DDOST or RPN1 (data not shown). We then examined the effect of NGI-1, a recently discovered N-linked glycosylation inhibitor that directly and reversibly interacts with the OST catalytic subunits (Lopez-Sambrooks et al., 2016). NGI-1 strongly impaired DENV and ZIKV NS1 glycosylation in a dose dependent manner (data not shown). We noticed that hypoglycosylated NS1 levels were reduced when compared to the fully glycosylated NS1 (data not shown). To pinpoint the effect of hypoglycosylation on NS1 stability, we transfected HEK-293T cells with plasmids encoding FLAG-NS1 WT or mutants lacking the first (N130Q), the second (N207Q), or both N-linked glycosylation sites (N130Q/N207Q) (data not shown). Immunoblotting of cell lysates showed a gradual reduction of NS1 expression that correlated with the degree of glycosylation. In contrast, the treatment of cellular lysates containing FLAG-NS1 with the N-Glycosidase F (PNGase F) did not reduce protein expression, suggesting that cellular processes might drive NS1 instability. To test whether hypoglycosylated NS1 is degraded by the proteasome, we transfected cells with plasmids encoding for WT or the N130Q/N207Q NS1 in the presence or absence of the proteasome inhibitor MG132. Western blots showed that NS1 N130Q/N207Q expression increased upon MG132 treatment (data not shown). Collectively, these results demonstrate that the OST complex and its catalytic activity are essential for NS1 glycosylation, which might be important for the proper folding and stability of the protein. Importantly, NGI-1 significantly inhibited DENV infection in a dose dependent manner (FIG. 2A). NGI-1 blocked vRNA amplification (FIG. 2B) and impaired NS1 secretion from infected cells (FIG. 2C). NGI-1 antiviral activity was comparable to mycophenolic acid (MPA), a well-known DENV replication inhibitor targeting the de novo purine biosynthesis pathway (Diamond et al., 2002) (FIGS. 2A and B). NGI-1 blocked DENV infection without any significant cytotoxicity (data not shown) or antiproliferative effects (FIG. 3). NGI-1 also blocked infection by the four DENV serotypes, WNV, and to a lesser extent ZIKV but not CHIKV or HSV (FIG. 2D). Furthermore, it significantly reduced DENV infection of human primary skin fibroblasts as well as monocyte-derived dendritic cells (FIG. 2E). Together, these results highlight an essential role of OST complex catalytic activity during DENV replication.

### REFERENCES

Throughout this application, various references describe the state of the art to which this invention pertains. The disclosures of these references are hereby incorporated by reference into the present disclosure.
Acosta, E. G., Kumar, A., and Bartenschlager, R. (2014). Revisiting dengue virus-host cell interaction: new insights into molecular and cellular virology. Adv. Virus Res. 88, 1-109.
Adams, D. R., Ron, D., and Kiely, P. A. (2011). RACK1, A multifaceted scaffolding protein: Structure and function. Cell Commun. Signal. CCS 9, 22.
Aguirre, S., Luthra, P., Sanchez-Aparicio, M. T., Maestre, A. M., Patel, J., Lamothe, F., Fredericks, A. C., Tripathi, S., Zhu, T., Pintado-Silva, J., et al. (2017). Dengue virus NS2B protein targets cGAS for degradation and prevents mitochondrial DNA sensing during infection. Nat. Microbiol. 2, 17037.
Akey, D. L., Brown, W. C., Dutta, S., Konwerski, J., Jose, J., Jurkiw, T. J., DelProposto, J., Ogata, C. M., Skiniotis, G., Kuhn, R. J., et al. (2014). Flavivirus NS1 structures reveal surfaces for associations with membranes and the immune system. Science 343, 881-885.
Ansarah-Sobrinho, C., Nelson, S., Jost, C. A., Whitehead, S. S., and Pierson, T. C. (2008). Temperature-dependent production of pseudoinfectious dengue reporter virus particles by complementation. Virology 381, 67-74.
Apte-Sengupta, S., Sirohi, D., and Kuhn, R. J. (2014). Coupling of replication and assembly in flaviviruses. Curr. Opin. Virol. 9, 134-142.
Bhatt, S., Gething, P. W., Brady, O. J., Messina, J. P., Farlow, A. W., Moyes, C. L., Drake, J. M., Brownstein, J. S., Hoen, A. G., Sankoh, O., et al. (2013). The global distribution and burden of dengue. Nature 496, 504-507.
Capeding, M. R., Tran, N. H., Hadinegoro, S. R. S., Ismail, H. I. H. J. M., Chotpitayasunondh, T., Chua, M. N., Luong, C. Q., Rusmil, K., Wirawan, D. N., Nallusamy, R., et al. (2014). Clinical efficacy and safety of a novel tetravalent dengue vaccine in healthy children in Asia: a phase 3, randomised, observer-masked, placebo-controlled trial. Lancet Lond. Engl. 384, 1358-1365.
Carpp, L. N., Rogers, R. S., Moritz, R. L., and Aitchison, J. D. (2014). Quantitative Proteomic Analysis of Host-virus Interactions Reveals a Role for Golgi Brefeldin A Resistance Factor 1 (GBF1) in Dengue Infection. Mol. Cell. Proteomics MCP 13, 2836.
Carvajal, F., Vallejos, M., Walters, B., Contreras, N., Hertz, M. I., Olivares, E., Caceres, C. J., Pino, K., Letelier, A., Thompson, S. R., et al. (2016). Structural domains within the HIV-1 mRNA and the ribosomal protein S25 influence cap-independent translation initiation. FEBS J. 283, 2508-2527.
Chatel-Chaix, L., Fischl, W., Scaturro, P., Cortese, M., Kallis, S., Bartenschlager, M., Fischer, B., and Bartenschlager, R. (2015). A Combined Genetic-Proteomic Approach Identifies Residues within Dengue Virus NS4B Critical for Interaction with NS3 and Viral Replication. J. Virol. 89, 7170-7186.
Chatel-Chaix, L., Cortese, M., Romero-Brey, I., Bender, S., Neufeldt, C. J., Fischl, W., Scaturro, P., Schieber, N., Schwab, Y., Fischer, B., et al. (2016). Dengue Virus Perturbs Mitochondrial Morphodynamics to Dampen Innate Immune Responses. Cell Host Microbe 20, 342-356.
Cherepanova, N., Shrimal, S., and Gilmore, R. (2016). N-linked glycosylation and homeostasis of the endoplasmic reticulum. Curr. Opin. Cell Biol. 41, 57-65.
Courageot, M. P., Frenkiel, M. P., Dos Santos, C. D., Deubel, V., and Desprès, P. (2000). Alpha-glucosidase inhibitors reduce dengue virus production by affecting the initial steps of virion morphogenesis in the endoplasmic reticulum. J. Virol. 74, 564-572.
Crabtree, M. B., Kinney, R. M., and Miller, B. R. (2005). Deglycosylation of the NS1 protein of dengue 2 virus, strain 16681: construction and characterization of mutant viruses. Arch. Virol. 150, 771-786.
De Maio, F. A., Risso, G., Iglesias, N. G., Shah, P., Pozzi, B., Gebhard, L. G., Mammi, P., Mancini, E., Yanovsky, M. J., Andino, R., et al. (2016). The Dengue Virus NS5 Protein Intrudes in the Cellular Spliceosome and Modulates Splicing. PLoS Pathog. 12, e1005841.
Dechtawewat, T., Paemanee, A., Roytrakul, S., Songprakhon, P., Limjindaporn, T., Yenchitsomanus, P.-T., Saitornuang, S., Puttikhunt, C., Kasinrerk, W., Malasit, P., et al. (2016). Mass spectrometric analysis of host cell proteins interacting with dengue virus nonstructural protein 1 in dengue virus-infected HepG2 cells. Biochim. Biophys. Acta 1864, 1270-1280.
Diamond, M. S., Zachariah, M., and Harris, E. (2002). Mycophenolic acid inhibits dengue virus infection by preventing replication of viral RNA. Virology 304, 211-221.
Doolittle, J. M., and Gomez, S. M. (2011). Mapping protein interactions between Dengue virus and its human and insect hosts. PLoS Negl. Trop. Dis. 5, e954.
Edgil, D., Polacek, C., and Harris, E. (2006). Dengue virus utilizes a novel strategy for translation initiation when cap-dependent translation is inhibited. J. Virol. 80, 2976-2986.
Fernandez-Garcia, M.-D., Meertens, L., Bonazzi, M., Cossart, P., Arenzana-Seisdedos, F., and Amara, A. (2011). Appraising the roles of CBLL1 and the ubiquitin/proteasome system for flavivirus entry and replication. J. Virol. 85, 2980-2989.
Fischl, W., and Bartenschlager, R. (2013). High-throughput screening using dengue virus reporter genomes. Methods Mol. Biol. Clifton N.J. 1030, 205-219.
Gillespie, L. K., Hoenen, A., Morgan, G., and Mackenzie, J. M. (2010). The endoplasmic reticulum provides the membrane platform for biogenesis of the flavivirus replication complex. J. Virol. 84, 10438-10447.
Gutsche, I., Coulibaly, F., Voss, J. E., Salmon, J., d'Alayer, J., Ermonval, M., Larquet, E., Charneau, P., Krey, T., Mégret, F., et al. (2011). Secreted dengue virus nonstructural protein NS1 is an atypical barrel-shaped high-density lipoprotein. Proc. Natl. Acad. Sci. U.S.A. 108, 8003-8008.
Guzman, M. G., and Harris, E. (2015). Dengue. Lancet Lond. Engl. 385, 453-465.
Haffner, C., Frauli, M., Topp, S., Irmler, M., Hofmann, K., Regula, J. T., Bally-Cuif, L., and Haass, C. (2004). Nicalin and its binding partner Nomo are novel Nodal signaling antagonists. EMBO J. 23, 3041.
Hiraishi, H., Oatman, J., Haller, S. L., Blunk, L., McGivern, B., Morris, J., Papadopoulos, E., Gutierrez, W., Gordon, M., Bokhari, W., et al. (2014). Essential role of eIF5-mimic protein in animal development is linked to control of ATF4 expression. Nucleic Acids Res. 42, 10321-10330.
Holbrook, M. R. (2017). Historical Perspectives on Flavivirus Research. Viruses 9, 97.
Huang, D. W., Sherman, B. T., and Lempicki, R. A. (2009a). Bioinformatics enrichment tools: paths toward the comprehensive functional analysis of large gene lists. Nucleic Acids Res. 37, 1-13.
Huang, D. W., Sherman, B. T., and Lempicki, R. A. (2009b). Systematic and integrative analysis of large gene lists using DAVID bioinformatics resources. Nat. Protoc. 4, 44-57.
Jha, S., Rollins, M. G., Fuchs, G., Procter, D. J., Hall, E. A., Cozzolino, K., Sarnow, P., Savas, J. N., and Walsh, D. (2017). Trans-kingdom mimicry underlies ribosome customization by a poxvirus kinase. Nature 546, 651-655.
Khadka, S., Vangeloff, A. D., Zhang, C., Siddavatam, P., Heaton, N. S., Wang, L., Sengupta, R., Sahasrabudhe, S., Randall, G., Gribskov, M., et al. (2011). A physical interaction network of dengue virus and human proteins. Mol. Cell. Proteomics MCP 10, M111.012187.
Kozel, C., Thompson, B., Hustak, S., Moore, C., Nakashima, A., Singh, C. R., Reid, M., Cox, C., Papadopoulos, E., Luna, R. E., et al. (2016). Overexpression of eIF5 or its protein mimic 5MP perturbs eIF2 function and induces ATF4 translation through delayed re-initiation. Nucleic Acids Res. 44, 8704-8713.
Kyle, J. L., and Harris, E. (2008). Global spread and persistence of dengue. Annu. Rev. Microbiol. 62, 71-92.
Landry, D. M., Hertz, M. I., and Thompson, S. R. (2009). RPS25 is essential for translation initiation by the Dicistroviridae and hepatitis C viral IRESs. Genes Dev. 23, 2753-2764.
Le Breton, M., Meyniel-Schicklin, L., Deloire, A., Coutard, B., Canard, B., de Lamballerie, X., Andre, P., Rabourdin-Combe, C., Lotteau, V., and Davoust, N. (2011). Flavivirus NS3 and NS5 proteins interaction network: a high-throughput yeast two-hybrid screen. BMC Microbiol. 11, 234.
Lee, E.-Y., Lee, H.-C., Kim, H.-K., Jang, S. Y., Park, S.-J., Kim, Y.-H., Kim, J. H., Hwang, J., Kim, J.-H., Kim, T.-H., et al. (2016). Infection-specific phosphorylation of glutamyl-prolyl tRNA synthetase induces antiviral immunity. Nat. Immunol. 17, 1252-1262.
Limjindaporn, T., Wongwiwat, W., Noisakran, S., Srisawat, C., Netsawang, J., Puttikhunt, C., Kasinrerk, W., Avirutnan, P., Thiemmeca, S., Sriburi, R., et al. (2009). Interaction of dengue virus envelope protein with endoplasmic reticulum-resident chaperones facilitates dengue virus production. Biochem. Biophys. Res. Commun. 379, 196-200.
Lindenbach, B. D., and Rice, C. M. (1997). trans-Complementation of yellow fever virus NS1 reveals a role in early RNA replication. J. Virol. 71, 9608-9617.
Lindenbach, B. D., and Rice, C. M. (1999). Genetic interaction of flavivirus nonstructural proteins NS1 and NS4A as a determinant of replicase function. J. Virol. 73, 4611-4621.
Lopez-Sambrooks, C., Shrimal, S., Khodier, C., Flaherty, D. P., Rinis, N., Charest, J. C., Gao, N., Zhao, P., Wells, L., Lewis, T. A., et al. (2016). Oligosaccharyltransferase inhibition induces senescence in RTK-driven tumor cells. Nat. Chem. Biol. 12, 1023-1030.
Mackenzie, J. M., Jones, M. K., and Young, P. R. (1996). Immunolocalization of the dengue virus nonstructural glycoprotein NS1 suggests a role in viral RNA replication. Virology 220, 232-240.
Mairiang, D., Zhang, H., Sodja, A., Murali, T., Suriyaphol, P., Malasit, P., Limjindaporn, T., and Finley, R. L. (2013). Identification of new protein interactions between dengue fever virus and its hosts, human and mosquito. PloS One 8, e53535.
Majzoub, K., Hafirassou, M. L., Meignin, C., Goto, A., Marzi, S., Fedorova, A., Verdier, Y., Vinh, J., Hoffmann, J. A., Martin, F., et al. (2014). RACK1 controls IRES-mediated translation of viruses. Cell 159, 1086-1095.
Marceau, C. D., Puschnik, A. S., Majzoub, K., Ooi, Y. S., Brewer, S. M., Fuchs, G., Swaminathan, K., Mata, M. A., Elias, J. E., Sarnow, P., et al. (2016). Genetic dissection of Flaviviridae host factors through genome-scale CRISPR screens. Nature 535, 159-163.
Meertens, L., Carnec, X., Lecoin, M. P., Ramdasi, R., Guivel-Benhassine, F., Lew, E., Lemke, G., Schwartz, O., and Amara, A. (2012). The TIM and TAM families of phosphatidylserine receptors mediate dengue virus entry. Cell Host Microbe 12, 544-557.
Miller, S., and Krijnse-Locker, J. (2008). Modification of intracellular membrane structures for virus replication. Nat. Rev. Microbiol. 6, 363-374.
Mim, C., and Unger, V. M. (2012). Membrane curvature and its generation by BAR proteins. Trends Biochem. Sci. 37, 526-533.
Mondotte, J. A., Lozach, P.-Y., Amara, A., and Gamarnik, A. V. (2007). Essential role of dengue virus envelope protein N glycosylation at asparagine-67 during viral propagation. J. Virol. 81, 7136-7148.
Mukherjee, S., Pierson, T. C., and Dowd, K. A. (2014). Pseudo-infectious reporter virus particles for measuring antibody-mediated neutralization and enhancement of dengue virus infection. Methods Mol. Biol. Clifton N.J. 1138, 75-97.
Naik, N. G., and Wu, H.-N. (2015). Mutation of Putative N-Glycosylation Sites on Dengue Virus NS4B Decreases RNA Replication. J. Virol. 89, 6746-6760.
Nakatani, Y., and Ogryzko, V. (2003). Immunoaffinity Purification of Mammalian Protein Complexes. B.-M. in Enzymology, ed. (Academic Press), pp. 430-444.
Nielsen, M. H., Flygaard, R. K., and Jenner, L. B. (2017). Structural analysis of ribosomal RACK1 and its role in translational control. Cell. Signal.
Nilsson, J., Sengupta, J., Frank, J., and Nissen, P. (2004). Regulation of eukaryotic translation by the RACK1 protein: a platform for signalling molecules on the ribosome. EMBO Rep. 5, 1137-1141.
Olagnier, D., Peri, S., Steel, C., van Montfoort, N., Chiang, C., Beljanski, V., Slifker, M., He, Z., Nichols, C. N., Lin, R., et al. (2014). Cellular oxidative stress response controls the antiviral and apoptotic programs in dengue virus-infected dendritic cells. PLoS Pathog. 10, e1004566.
Onoguchi, K., Onomoto, K., Takamatsu, S., Jogi, M., Takemura, A., Morimoto, S., Julkunen, I., Namiki, H., Yoneyama, M., and Fujita, T. (2010). Virus-infection or 5′ppp-RNA activates antiviral signal through redistribution of IPS-1 mediated by MFN1. PLoS Pathog. 6, e1001012.
Poyomtip, T., Hodge, K., Matangkasombut, P., Sakuntabhai, A., Pisitkun, T., Jirawatnotai, S., and Chimnaronk, S. (2016). Development of viable TAP-tagged dengue virus for investigation of host-virus interactions in viral replication. J. Gen. Virol. 97, 646-658.
Pryor, M. J., and Wright, P. J. (1994). Glycosylation mutants of dengue virus NS1 protein. J. Gen. Virol. 75 (Pt 5), 1183-1187.
Pryor, M. J., Gualano, R. C., Lin, B., Davidson, A. D., and Wright, P. J. (1998). Growth restriction of dengue virus type 2 by site-specific mutagenesis of virus-encoded glycoproteins. J. Gen. Virol. 79 (Pt 11), 2631-2639.
Roboti, P., and High, S. (2012). The oligosaccharyltransferase subunits OST48, DAD1 and KCP2 function as ubiquitous and selective modulators of mammalian N-glycosylation. J. Cell Sci. 125, 3474-3484.
Roth, H., Magg, V., Uch, F., Mutz, P., Klein, P., Haneke, K., Lohmann, V., Bartenschlager, R., Fackler, O. T., Locker, N., et al. (2017). Flavivirus Infection Uncouples Translation Suppression from Cellular Stress Responses. MBio 8.
Ruiz-Canada, C., Kelleher, D. J., and Gilmore, R. (2009). Cotranslational and posttranslational N-glycosylation of polypeptides by distinct mammalian OST isoforms. Cell 136, 272-283.
Savidis, G., McDougall, W. M., Meraner, P., Perreira, J. M., Portmann, J. M., Trincucci, G., John, S. P., Aker, A. M., Renzette, N., Robbins, D. R., et al. (2016). Identification of Zika Virus and Dengue Virus Dependency Factors using Functional Genomics. Cell Rep. 16, 232-246.
Scaturro, P., Cortese, M., Chatel-Chaix, L., Fischl, W., and Bartenschlager, R. (2015). Dengue Virus Non-structural Protein 1 Modulates Infectious Particle Production via Interaction with the Structural Proteins. PLoS Pathog. 11, e1005277.
Shannon, P., Markiel, A., Ozier, O., Baliga, N. S., Wang, J. T., Ramage, D., Amin, N., Schwikowski, B., and Ideker, T. (2003). Cytoscape: a software environment for integrated models of biomolecular interaction networks. Genome Res. 13, 2498-2504.
Shrimal, S., Cherepanova, N. A., and Gilmore, R. (2015). Cotranslational and posttranslocational N-glycosylation of proteins in the endoplasmic reticulum. Semin. Cell Dev. Biol. 41, 71-78.
Somnuke, P., Hauhart, R. E., Atkinson, J. P., Diamond, M. S., and Avirutnan, P. (2011). N-linked glycosylation of dengue virus NS1 protein modulates secretion, cell-surface expression, hexamer stability, and interactions with human complement. Virology 413, 253-264.
Sun, B., Sundstrom, K. B., Chew, J. J., Bist, P., Gan, E. S., Tan, H. C., Goh, K. C., Chawla, T., Tang, C. K., and Ooi, E. E. (2017). Dengue virus activates cGAS through the release of mitochondrial DNA. Sci. Rep. 7, 3594.
Tajima, S., Takasaki, T., and Kurane, I. (2008). Characterization of Asn130-to-Ala mutant of dengue type 1 virus NS1 protein. Virus Genes 36, 323-329.
Warde-Farley, D., Donaldson, S. L., Comes, O., Zuberi, K., Badrawi, R., Chao, P., Franz, M., Grouios, C., Kazi, F., Lopes, C. T., et al. (2010). The GeneMANIA prediction server: biological network integration for gene prioritization and predicting gene function. Nucleic Acids Res. 38, W214-220.
Watterson, D., Modhiran, N., and Young, P. R. (2016). The many faces of the flavivirus NS1 protein offer a multitude of options for inhibitor design. Antiviral Res. 130, 7-18.
Wei, Y., Chiang, W.-C., Sumpter, R., Mishra, P., and Levine, B. (2017). Prohibitin 2 Is an Inner Mitochondrial Membrane Mitophagy Receptor. Cell 168, 224-238.e10.
Welsch, S., Miller, S., Romero-Bret′, I., Merz, A., Bleck, C. K. E., Walther, P., Fuller, S. D., Antony, C., Krijnse-Locker, J., and Bartenschlager, R. (2009). Composition and three-dimensional architecture of the dengue virus replication and assembly sites. Cell Host Microbe 5, 365-375.
West, A. P., Shadel, G. S., and Ghosh, S. (2011). Mitochondria in innate immune responses. Nat. Rev. Immunol. 11, 389-402.
Westaway, E. G., Mackenzie, J. M., Kenney, M. T., Jones, M. K., and Khromykh, A. A. (1997). Ultrastructure of Kunjin virus-infected cells: colocalization of NS1 and NS3 with double-stranded RNA, and of NS2B with NS3, in virus-induced membrane structures. J. Virol. 71, 6650-6661.
Xu, Z., Anderson, R., and Hobman, T. C. (2011). The Capsid-Binding Nucleolar Helicase DDX56 Is Important for Infectivity of West Nile Virus. J. Virol. 85, 5571-5580.
Youn, S., Li, T., McCune, B. T., Edeling, M. A., Fremont, D. H., Cristea, I. M., and Diamond, M. S. (2012). Evidence for a genetic and physical interaction between nonstructural proteins NS1 and NS4B that modulates replication of West Nile virus. J. Virol. 86, 7360-7371.
Youn, S., Ambrose, R. L., Mackenzie, J. M., and Diamond, M. S. (2013). Non-structural protein-1 is required for West Nile virus replication complex formation and viral RNA synthesis. Virol. J. 10, 339.
Zeidler, J. D., Fernandes-Siqueira, L. O., Barbosa, G. M., and Da Poian, A. T. (2017). Non-Canonical Roles of Dengue Virus Non-Structural Proteins. Viruses 9.
Zhang, R., Miner, J. J., Gorman, M. J., Rausch, K., Ramage, H., White, J. P., Zuiani, A., Zhang, P., Fernandez, E., Zhang, Q., et al. (2016). A CRISPR screen defines a signal peptide processing pathway required by flaviviruses. Nature 535, 164-168.

1-5. (canceled)

6. A method for treating Dengue virus infection comprising administering to a subject in need thereof a therapeutically effective amount of an inhibitor of one or more of: OST complex, CCT complex and RACK1.

7. The method of claim 6, wherein the inhibitor is an inhibitor of the OST complex.

8. The method of claim 6, wherein the inhibitor is a small organic molecule, an antibody or a polypeptide.

9. The method of claim 6, wherein the inhibitor is NGI-1.
